(19) United States
(12) Patent Application Publication
(10) Pub. No.: US 2010/0261945 A1
(43) Pub. Date: Oct. 14, 2010

(54) METHOD OF SEPARATING AROMATIC COMPOUND FROM MIXTURE CONTAINING AROMATIC COMPOUND AND ALIPHATIC COMPOUND

(75) Inventors: Myong Hoon LIM, Yongin-si (KR); Young Mi Kim, Yongin-si (KR); Yong Soo Kim, Suwon-si (KR); Jae Eun Rho, Yongin-si (KR); Hyun Woo, Seongnam-si (KR); Young Youl Koo, Seoul (KR)

Correspondence Address: BUCHANAN, INGERSOLL & ROONEY PC, POST OFFICE BOX 1404, ALEXANDRIA, VA, 22313-1404, US

(73) Assignee: Samsung Engineering Co., Ltd., Gangnam-gu (KR)

(21) Appl. No.: 12/755,951

(22) Filed: Apr. 7, 2010

(30) Foreign Application Priority Data
Apr. 8, 2009 (KR) 10-2009-0030517
May 19, 2009 (KR) 10-2009-0043599

(51) Int. Cl. C07C 7/10

(52) U.S. Cl. 585/865; 585/833; 585/864

(57) ABSTRACT

Provided is a method of separating an aromatic compound from a mixture containing the aromatic compound and an aliphatic compound. The method includes separating an aromatic compound from a mixture containing the aromatic compound and an aliphatic compound, wherein the separating is performed using a liquid-liquid extracting method using an ionic liquid as an extracting solvent.

### CROSS-REFERENCE TO RELATED PATENT APPLICATIONS

This application claims the benefit of Korean Patent Application No. 10-2009-0030517, filed on Apr. 08, 2009 and Korean Patent Application No. 10-2009-0043599, filed on May 19, 2009, in the Korean Intellectual Property Office, the disclosures of which are incorporated herein in their entirety by reference.

### BACKGROUND OF THE INVENTION

1. Field of the Invention

The present invention relates to a method of separating an aromatic compound from a mixture containing the aromatic compound and an aliphatic compound, and more particularly, to a method of separating an aromatic compound from a mixture containing the aromatic compound and an aliphatic compound, wherein the separating is performed using a liquid-liquid extracting method using an ionic liquid as an extracting solvent.

2. Description of the Related Art

Ionic compounds such as salt generally have a high melting point of at least 800° C., whereas ionic liquids have a low melting point of 100° C. or less, despite the fact that ionic liquids include cations and anions. Besides the low melting point, ionic liquids are nonvolatile, noncombustible, and have high thermal stability, and thus are used as reaction solvents or catalysts. In this regard, ionic liquids have higher selectivity and reactivity compared to organic solvents that are formed of monomolecule. In addition, ionic liquids used as reaction solvents or catalysts may be reused, and thus are environmental friendly materials.

Meanwhile, naphtha that is generated during purifying of petroleum includes an aromatic compound, such as benzene, toluene, xylene, and ethyl benzene; and an aliphatic compound, such as butane, pentane, cyclopentane, heptane, and octane. The aromatic compound and the aliphatic compound are separated from each other and each used in various fields.

Currently, a polar solvent, such as sulfolane, N-methylpirrolidone, N-formyl morpholine, dimethylsulfoxide (Emmrich, G., et al., Hydrocarbon Technol. Int. 161-165 (1990)), is used as an extracting solvent when separating the aromatic compound from the naphtha. However, some of these polar solvents may dissolve into the aliphatic compound during liquid-liquid extraction, and thus a separate process of removing the polar solvents from the aliphatic compound may be required.

### SUMMARY OF THE INVENTION

The present invention provides a method of separating an aromatic compound from a mixture containing the aromatic compound and an aliphatic compound, wherein the separating is performed using a liquid-liquid extracting method using an ionic liquid as an extracting solvent.

According to an aspect of the present invention, there is provided a method of separating an aromatic compound, the method including separating an aromatic compound from a mixture containing the aromatic compound and an aliphatic compound, wherein the separating is performed using a liquid-liquid extracting method using an ionic liquid as an extracting solvent.

The ionic liquid may include at least one type of compound selected from compounds represented by Formula 1 below:

(Cat₁⁺)(An₁⁻) (Formula 1)

Here, Cat₁⁺ may include a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, wherein each cation may include at least one substituted or unsubstituted C₂-C₂₀ alkenyl group; and An₁⁻ may include an anion including at least one halogen atom.

Cat₁⁺ may include a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, wherein each cation may include at least one group from the group consisting of a substituted or unsubstituted allyl group and vinyl group.

Cat₁⁺ may include a quaternary ammonium cation or quaternary phosphonium cation represented by Formula 2 below:

(R₁R₂R₃R₄Z⁺) (Formula 2)

Here, Z may include nitrogen or phosphorous and R₁, R₂, R₃, and R₄ are each independently selected from the group consisting of a substituted or unsubstituted C₁-C₁₀ alkyl group, a substituted or unsubstituted C₃-C₁₀ cycloalkyl group, a substituted or unsubstituted C₂-C₂₀ alkenyl group, and a substituted or unsubstituted C₆-C₂₀ aryl group, wherein at least one of R₁, R₂, R₃, and R₄ may include at least one group selected from the group consisting of a substituted or unsubstituted allyl group and a vinyl group.

An₁⁻ may include an anion selected from the group consisting of F⁻, Cl⁻, Br⁻, I⁻, BF₄⁻, PF₆⁻, SbF₆⁻, AsF₆⁻, CF₃SO₃⁻, CF₃COO⁻, HCF₂CF₂SO₃⁻, CF₃HFCCF₂SO₃⁻, HCClFCF₂SO₃⁻, CF₃OCFHCF₂SO₃—, CF₃CF₂OCFHCF₂SO₃⁻, CF₃CFHOCF₂CF₂SO₃⁻, CF₂HCF₂OCF₂CF₂SO₃⁻, CF₂ICF₂OCF₂CF₂SO₃⁻, CF₃CF₂OCF₂CF₂SO₃⁻, (CF₂HCF₂SO₂)₂N⁻, (CF₃CFHCF₂SO₂)₂N⁻, CF₃CH₂CH₂COO⁻, (CF₃SO₂)₂N⁻, (CF₃SO₂)₂C⁻, (FSO₂)₂N⁻, (CF₃CF₂)₂(SO₂)₂N⁻, and PF₃(C₂F₅)₃⁻.

The ionic liquid may include at least one selected from the group consisting of 1-allyl-3-butyl imidazolium bromide, 1-allyl-3-butyl imidazolium chloride, 1-allyl-3-butyl imidazolium tetrafluoroborate, 1-allyl-3-butyl imidazolium hexafluorophosphate, 1-allyl-3-butyl imidazolium bis(trifluorosulfonyl)imide, 1,3-diallyl imidazolium bromide, 1,3-diallyl imidazolium chloride, 1,3-diallyl imidazolium tetrafluoroborate, 1,3-diallyl imidazolium hexafluoro phosphate, 1,3-diallyl imidazolium bis(trifluorosulfonyl)imide, 1-allyl-3-vinyl imidazolium bromide, 1-allyl-3-vinyl imidazolium chloride, 1-allyl-3-vinyl imidazolium tetrafluoroborate, 1-allyl-3-vinyl imidazolium hexafluoro phosphate, 1-allyl-3-vinyl imidazolium bis(trifluorosulfonyl)imide, 1-butyl-3-vinyl imidazolium bromide, 1-butyl-3-vinyl imidazolium chloride, 1-butyl-3-vinyl imidazolium tetrafluoroborate, 1-butyl-3-vinyl imidazolium hexafluoro phosphate, and 1-butyl-3-vinyl imidazolium bis(trifluorosulfonyl)imide.

The ionic liquid may additionally include at least one compound selected from compounds represented by Formula 1′ below:

(Cat₂⁺)(An₂⁻) (1′)

Here, Cat₂⁺ may include a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, and An₂⁻ may include an anion comprising at least one halogen atom. For example, An₂⁻ may be identical to An₁⁻ above.

The mixture may include at least 1 molar part of the aliphatic compound with respect to 100 molar parts of the aromatic compound.

### BRIEF DESCRIPTION OF THE DRAWINGS

The above and other features and advantages of the present invention will become more apparent by describing in detail exemplary embodiments thereof with reference to the attached drawings in which:

FIG. 1 is a triangular phase diagram showing a result of separating toluene from heptane-toluene mixtures having various compositions by using 1-allyl-3-butyl imidazolium bromide, which is an ionic liquid, as an extracting solvent, at a temperature of 25° C. and a pressure of 1 atm (Examples 1-1 through 1-9);

FIG. 2 is a triangular phase diagram showing a result of separating toluene from heptane-toluene mixtures having various compositions by using 1-allyl-3-butyl imidazolium bromide, which is an ionic liquid, as an extracting solvent, at a temperature of 40° C. and a pressure of 1 atm (Examples 2-1 through 2-9);

FIG. 3 is a triangular phase diagram showing a result of separating toluene from heptane-toluene mixtures having various compositions by using 1-allyl-3-butyl imidazolium bromide, which is an ionic liquid, as an extracting solvent, at a temperature of 60° C. and a pressure of 1 atm (Examples 3-1 through 3-6);

FIG. 4 is a triangular phase diagram showing a result of separating toluene from heptane-toluene mixtures having various compositions by using 1-allyl-3-butyl imidazolium tetrafluoroborate, which is an ionic liquid, as an extracting solvent, at a temperature of 25° C. and a pressure of 1 atm (Examples 4-1 through 4-5);

FIG. 5 is a triangular phase diagram showing a result of separating toluene from heptane-toluene mixtures having various compositions by using 1,3-diallyl imidazolium bromide, which is an ionic liquid, as an extracting solvent, at a temperature of 25° C. and a pressure of 1 atm (Examples 5-1 through 5-8); and

FIG. 6 is a triangular phase diagram showing a result of separating toluene from heptanes-toluene mixtures having various compositions by using a mixture of 80 molar parts of 1-allyl-3-butyl-imidazolium bromide and 20 molar parts of 1-butyl-3-methyl imidazolium chloride, which are ionic liquids, as an extracting solvent, at a temperature of 25° C. and a pressure of 1 atm (Examples 6-1 through 6-3).

FIG. 7 is a triangular phase diagram showing a result of separating toluene from heptanes-toluene mixtures having various compositions by using a mixture of 1-butyl-3-propyl imidazolium bromide, which is an ionic liquid, as an extracting solvent, at a temperature of 25° C. and a pressure of 1 atm (Examples 7-1 through 7-6).

### DETAILED DESCRIPTION OF THE INVENTION

Hereinafter, the present invention will be described more fully with reference to the accompanying drawings, in which exemplary embodiments of the invention are shown.

A method of separating an aromatic compound according to an embodiment of the present invention includes separating an aromatic compound from a mixture containing the aromatic compound and an aliphatic compound, wherein the separating is performed using a liquid-liquid extracting method using an ionic liquid as an extracting solvent.

In the present application, the aromatic compound denotes benzene or a derivative thereof, which is an organic compound having a benzene ring in molecules, and may be toluene. Also in the present application, the aliphatic compound denotes an organic compound which does not contain benzine rings, and may be heptane.

The mixture, for example, may include at least 1 molar part of the aliphatic compound with respect to 100 molar parts of the aromatic compound. When the amount of the aliphatic compound is less than 1 molar part with respect to 100 molar parts of the aromatic compound in the mixture, a lot of ionic liquid is required to extract the aromatic compound, and thus it is not economical. When the amount of the aliphatic compound increases in the mixture, the aromatic compound may be more easily removed.

Affinity of the aromatic compound to the ionic liquid is higher than affinity of the aromatic compound to the aliphatic compound. Accordingly, when the ionic liquid contacts the mixture containing the aromatic compound and the aliphatic compound (such a process is called an extracting operation), a massive amount of the aromatic compound contained in the mixture is extracted by being dissolved in the ionic liquid, and the aliphatic compound remains without being extracted. Also, most of the aromatic compound is separated from the mixture during the extracting operation, and thus the aromatic compound can be highly purified via a separate refining process for separating the aromatic compound from the ionic liquid.

The method according to the present embodiment has high separating efficiency, may be successfully performed at room temperature, and continuously reuses the ionic liquid, i.e. the extracting solvent, by recycling the ionic liquid. Accordingly, the method is economical and environmentally friendly. In other words, the ionic liquid may not only replace a polar organic solvent, such as sulfolane, N-methyl pyrrolidone, N-formyl morpholine, and/or dimethylsulfoxide, which is conventionally used as an extracting solvent for separating an aromatic compound, but also provide higher separating efficiency than the polar organic solvent. Also, since some of the polar organic solvent may dissolve into the aliphatic compound during an extracting operation, a method of separating an aromatic compound using a polar organic solvent as an extracting solvent requires a separate refining process so as to remove the polar organic solvent from the aliphatic compound after the extracting operation. However, since the ionic liquid barely dissolves in the aliphatic compound during an extracting operation, the method of separating an aromatic compound using the ionic liquid as an extracting solvent according to the present embodiment does not require a separate refining process for separating the ionic liquid from the aliphatic compound after the extracting operation.

The ionic liquid, for example, may include at least one type of compound selected from compounds represented by Formula 1 below.

(Cat₁⁺)(An₁⁻) (Formula 1)

Here, Cat₁⁺ may be a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, wherein each cation includes at least one substituted or unsubstituted C₂-C₂₀ alkenyl group; and An₁⁻ may be an anion including at least one halogen atom. Also, the substituted or unsubstituted C₂-C₂₀ alkenyl group may be a substituted or unsubstituted allyl group and/or vinyl group.

In the present application, the term “substitute” means that hydrogen is substituted with a substituent such as a halogen group, a hydroxyl group, an alkyl group, an alkoxy group, an amine group, or a mixed group thereof.

Cat₁⁺, may be, for example, a quaternary ammonium cation or a quaternary phosphonium cation represented by Formula 2 below.

(R₁R₂R₃R₄Z⁺) (Formula 2)

In Formula 2, Z may be nitrogen or phosphorus and R₁, R₂, R₃, and R₄ may be each independently selected from the group consisting of a substituted or unsubstituted C₁-C₁₀ alkyl group (for example, C₁-C₈ alkyl group), a substituted or unsubstituted C₃-C₁₀ cycloalkyl group (for example, C₃-C₈ cycloalkyl group), a substituted or unsubstituted C₂-C₂₀ alkenyl group, and a substituted or unsubstituted C₆-C₂₀ aryl group. Here, at least one of R₁, R₂, R₃, and R₄ may include at least one group selected from the group consisting of a substituted or unsubstituted allyl group and a vinyl group.

An₁⁻, for example, may be F⁻, Cl⁻, Br⁻, I⁻, BF₄⁻, PF₆⁻, SbF₆⁻, AsF₆⁻, CF₃SO₃⁻, CF₃COO⁻, HCF₂CF₂SO₃⁻, CF₃HFCCF₂SO₃⁻, HCClFCF₂SO₃⁻, CF₃OCFHCF₂SO₃—, CF₃CF₂OCFHCF₂SO₃⁻, CF₃CFHOCF₂CF₂SO₃⁻, CF₂HCF₂OCF₂CF₂SO₃⁻, CF₂ICF₂OCF₂CF₂SO₃⁻, CF₃CF₂OCF₂CF₂SO₃⁻, (CF₂HCF₂SO₂)₂N⁻, (CF₃CFHCF₂SO₂)₂N⁻, CF₃CH₂CH₂COO⁻, (CF₃SO₂)₂N⁻, (CF₃SO₂)₂C⁻, (FSO₂)₂N⁻, (CF₃CF₂)₂(SO₂)₂N⁻, or PF₃(C₂F₅)₃⁻.

The ionic liquid, for example, may include at least one selected from the group consisting of 1-allyl-3-butyl imidazolium bromide, 1-allyl-3-butyl imidazolium chloride, 1-allyl-3-butyl imidazolium tetrafluoroborate, 1-allyl-3-butyl imidazolium hexafluoro phosphate, 1-allyl-3-butyl imidazolium bis(trifluorosulfonyl)imide, 1,3-diallyl imidazolium bromide, 1,3-diallyl imidazolium chloride, 1,3-diallyl imidazolium tetrafluoroborate, 1,3-diallyl imidazolium hexafluoro phosphate, 1,3-diallyl imidazolium bis(trifluorosulfonyl)imide, 1-allyl-3-vinyl imidazolium bromide, 1-allyl-3-vinyl imidazolium chloride, 1-allyl-3-vinyl imidazolium tetrafluoroborate, 1-allyl-3-vinyl imidazolium hexafluoro phosphate, 1-allyl-3-vinyl imidazolium bis(trifluorosulfonyl)imide, 1-butyl-3-vinyl imidazolium bromide, 1-butyl-3-vinyl imidazolium chloride, 1-butyl-3-vinyl imidazolium tetrafluoroborate, 1-butyl-3-vinyl imidazolium hexafluoro phosphate, and 1-butyl-3-vinyl imidazolium bis(trifluorosulfonyl)imide.

Some of the ionic liquids described above may be represented by any one of the following formulas.

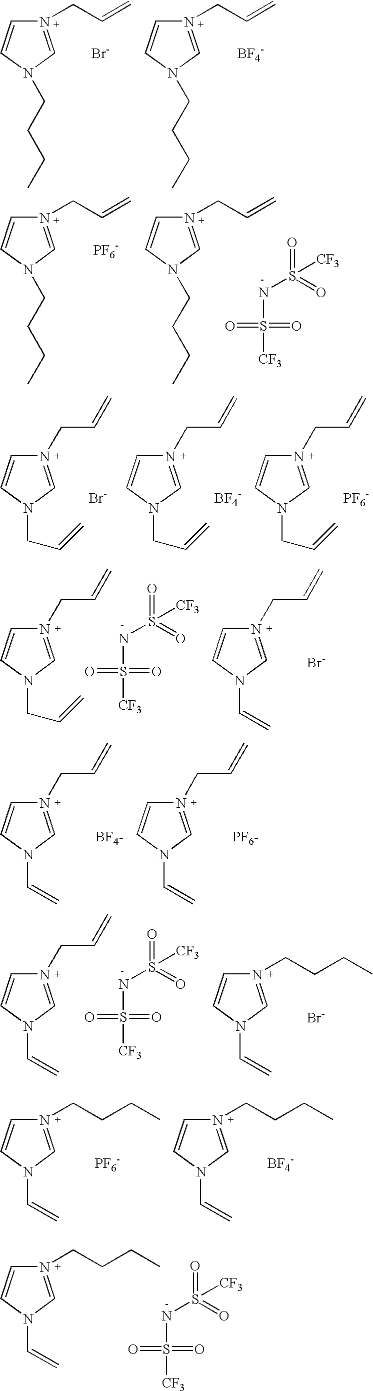

Meanwhile, the ionic liquid may include 1-butyl-3-propyl imidazolium bromide represented by the following formula.

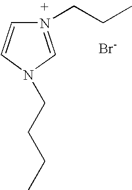

In this case, the ionic liquid may additionally include at least one compound selected from compounds represented by Formula 1′ below.

(Cat₂⁺)(An₂⁻) (Formula 1′)

Here, Cat₂⁺ may be a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, and An₂⁻ may be an anion including at least one halogen atom.

Also, An₂⁻ may be identical to An₁⁻ described above.

The ionic liquid has a melting point at 30° C. or less. Also, viscosity of the ionic liquid may be adjusted by mixing at least two different ionic liquid, thereby using an eutectic effect.

The present invention will be described in greater detail with reference to the following examples. The following examples are for illustrative purposes only and are not intended to limit the scope of the invention.

### EXAMPLES

### Examples 1-1 through 1-9

### Separation of toluene from mixture of toluene-heptane (1-allyl-3-butyl imidazolium bromide was used as an extracting solvent)

1) Step 1: Preparation of Mixtures of toluene-heptane

20 mL of each of 9 types of mixtures were prepared by mixing toluene and heptane in mole ratios of 1:9, 2:8, 3:7, 4:6, 5:5, 6:4, 7:3, 8:2, and 9:1, respectively.

2) Step 2: Preparation of 1-allyl-3-butyl imidazolium bromide

0.81 mol of 1-butylimidazole was mixed with 150 mL of acetone (constituting a solvent) at 0° C., and the mixture was stirred. 0.81 mol of allyl bromide was slowly added to the mixture of butylimidazole and acetone, and the mixture was stirred for 1 hour at 0° C. to initiate a reaction. Then, the reaction was performed for 24 hours at room temperature, and the remaining acetone was removed by using a vacuum distillation evaporator (Buchi, Rotavapor R-124). Then, the resultant was dried by using a vacuum pump for 24 hours so as to obtain an ionic liquid having a red color.

A result of nuclear magnetic resonance (NMR) analysis of the ionic liquid was as follows.

Result of NMR Analysis: 1H NMR (CDCl₃, 400 MHz) 9.89 (s, 1H), 7.42 (d, 1H), 7.30 (d, 1H), 5.64 (m, 1H), 5.07 (dd, 1H), 5.02 (dd, 1H), 4.66 (d, 2H), 3.98 (t, 2H), 1.51 (q, 2H), 0.97 (m, 2H), 0.52 (t, 3H)

3) Step 3: Separation of Toluene

20 mL of one of the mixtures prepared in Step 1 and 20 mL of the ionic liquid prepared in Step 2 were injected into a separatory funnel and then mixed together. Then, the separatory funnel was put into a thermostat maintained at a temperature of 25° C. and a pressure of 1 atm, and stabilized for 24 hours, so as to separate the resultant into a heptane layer and a toluene-containing ionic liquid layer.

### Examples 2-1 through 2-9

### Separation of toluene from mixture of toluene-heptane (1-allyl-3-butyl imidazolium bromide was used as an extracting solvent)

20 mL of each of 9 types of mixtures were prepared by mixing toluene and heptane in mole ratios of 1:9, 2:8, 3:7, 4:6, 5:5, 6:4, 7:3, 8:2, and 9:1, respectively. Toluene was separated in the same manner as in Examples 1-1 through 1-9, except that a separatory funnel was stabilized for 24 hours in a thermostat maintained at a temperature of 40° C. and a pressure of 1 atm so as to separate the resultant into a heptane layer and a toluene-containing ionic liquid layer.

### Examples 3-1 through 3-6

### Separation of toluene from mixture of toluene-heptane (1-allyl-3-butyl imidazolium bromide was used as an extracting solvent)

20 mL of each of 6 types of mixtures were prepared by mixing toluene and heptane in mole ratios of 2:8, 3:7, 4:6, 5:5, 6:4, and 7:3. Toluene was separated in the same manner as in Examples 1-1 through 1-9, except that a separatory funnel was stabilized for 24 hours in a thermostat maintained at a temperature of 60° C. and a pressure 1 atm so as to separate the resultant into a heptane layer and a toluene-containing ionic liquid layer.

### Examples 4-1 through 4-5

### Separation of toluene from mixture of toluene-heptane (1-allyl-3-butyl imidazolium tetrafluoroborate was used as an extracting solvent)

1) Step 1: Preparation of mixture of toluene-heptane

20 mL of each of 5 types of mixtures were prepared by mixing toluene and heptane in mole ratios of 3:7, 4:6, 5:5, 6:4, and 7:3, respectively.

2) Step 2: Preparation of 1-allyl-3-butyl imidazolium tetrafluoroborate

0.2 mol of 1-allyl-3-butyl imidazolium bromide prepared in Step 2 of Examples 1-1 through 1-9 was dissolved in 200 mL of distilled water so as to obtain a mixed solution. Viscous ionic liquid having a red color was obtained in the same manner as in Step 2 of Examples 1-1 through 1-9, except that 0.2 mol of sodium tetrafluoroborate was added to the mixed solution and was reacted for 24 hours.

A result of NMR analysis of the ionic liquid was as follows.

Result of NMR Analysis: 1H NMR (CDCl₃, 400 MHz) 8.80 (s, 1 H), 7.41 (d, 2H), 5.97 (m, 1H), 5.41 (m, 2H), 4.78 (d, 2H), 4.18 (t, 2H), 1.83 (q, 2H), 1.32 (m, 2H), 0.91 (t, 3H)

3) Step 3: Separation of toluene

Toluene was separated in the same manner as in Step 3 of Examples 1-1 through 1-9.

### Examples 5-1 through 5-8

### Separation of toluene from mixture of toluene-heptane (1,3-diallyl imidazolium bromide was used as an extracting solvent)

1) Step 1: Preparation of mixture of toluene-heptane

20 mL of each of 8 types of mixtures were prepared by mixing toluene and heptane in mole ratios of 2:8, 3:7, 4:6, 5:5, 6:4, 7:3, 8:2, and 9:1, respectively.

2) Step 2: Preparation of 1,3-diallyl imidazolium bromide

0.44 mol of imidazole and 200 mL of acetonitrile were mixed and then stirred at room temperature. 1.2 mol of potassium carbonate and 0.88 mol of allyl bromide were added to the mixture of imidazole and acetonitrile and then refluxed for 24 hours to form a reaction mixture. Then, solids were removed from the reaction mixture, and then the acetonitrile remaining in the reaction mixture was removed by using a vacuum distillation evaporator (Buchi, Rotavapor R-124) so as to obtain an ionic liquid. The ionic liquid was dried for 24 hours by using a vacuum pump so as to obtain a viscous ionic liquid having an orange color.

A result of NMR analysis of the ionic liquid was as follows.

Result of NMR Analysis: 1H NMR (CDCl₃, 400 MHz) 9.91 (s, 1H), 7.43 (s, 1H), 7.42 (s, 1H), 5.76 (m, 2H), 5.20 (m, 4H), 4.77 (m, 4H).

3) Step 3: Separation of toluene

Toluene was separated in the same manner as in Step 3 of Examples 1-1 through 1-9.

### Examples 6-1 through 6-3

### Separation of toluene from mixture of toluene-heptane (mixed ionic liquid of 1-allyl-3-butyl imidazolium bromide and 1-butyl-3-methyl imidazole chloride was used as an extracting solvent)

1) Step 1: Preparation of mixture of toluene-heptane

20 mL of each of 3 types of mixtures were prepared by mixing toluene and heptane in mole ratios of 3:7, 5:5, and 7:3, respectively.

2) Step 2: Preparation of mixed ionic liquid of 1-allyl-3-butyl imidazolium bromide and 1-butyl-3-methyl imidazole chloride

80 molar parts of 1-allyl-3-butyl imidazolium bromide prepared in Step 2 of Examples 1-1 through 1-9 and 20 molar parts of 1-butyl-3-methyl imidazole chloride (Fluka) were mixed, and the mixture thereof was dried for 24 hours by using a vacuum pump so as to obtain an ionic liquid.

3) Step 3: Separation of toluene

Toluene was separated in the same manner as in Step 3 of Examples 1-1 through 1-9.

### Examples 7-1-7-6

### Separation of toluene from mixture of toluene-heptane (1-butyl-3-propyl imidzolium bromide was used as an extracting solvent)

1) Step 1: Preparation of mixture of toluene-heptane

20 mL of each of 6 types of mixtures were prepared by mixing toluene and heptane in mole ratios of 2:8, 3:7, 4:6, 5:5, 6:4 and 7:3, respectively.

2) Step 2: Preparation of 1-butyl-3-propyl imidazolium bromide

0.81 mol of 1-butylimidazol was mixed with 200 mL of acetonitrile (constituting a solvent), and then the mixture was stirred at room temperature. 0.81 mol of propyl bromide was slowly added to the mixture, and then a reflux reaction was performed on the resultant at a temperature of 70° C. for 24 hours. After the reflux reaction, the remaining acetonitrile was removed by using a vacuum distillation evaporator (Buchi, Rotavapor R-124), and then the resultant was dried by using a vacuum pump for 24 hours so as to obtain an ionic liquid having a red color.

A result of NMR analysis of the ionic liquid was as follows.

Result of NMR Analysis: 1H NMR (CDCl₃, 400 MHz) 10.30 (s, 1H), 7.57 (m, 2H), 4.15 (m, 4H), 1.73 (m, 4H), 1.13 (m, 2H), 0.70 (m, 6H)

3) Step 3: Separation of Toluene

20 mL of one of the mixtures of toluene-heptane prepared in Step 1 and the ionic liquid prepared in Step 2 were injected into a separatory funnel and then mixed together. Then, the separatory funnel was put into a thermostat maintained at a temperature of 25° C. and a pressure of 1 atm, and stabilized for 24 hours, so as to separate the resultant into a heptane layer and a toluene-containing ionic liquid layer.

### Evaluation Example

### Evaluation of Separation Efficiency of Toluene

The heptane layer and the toluene-containing ionic liquid layer obtained in each example were then analyzed.

In this regard, the heptane layer and the toluene-containing ionic liquid layer were each analyzed by using NMR (Varian 400-MR), so as to measure a mole ratio of toluene and ionic liquid contained in each of the heptane layer and the toluene-containing ionic liquid layer.

Also, a mole ratio of heptane and toluene contained in the ionic liquid layer, and a mole ratio of heptane and toluene contained in the heptane layer were each measured by using a Gas Chromatography-Mass Spectroscopy (GC-MS) (Perkin Elmer-Clarus 600)-head space.

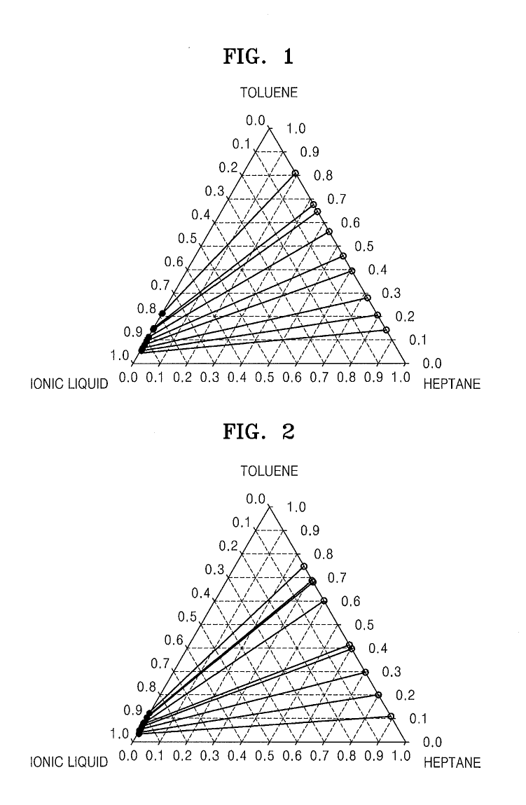

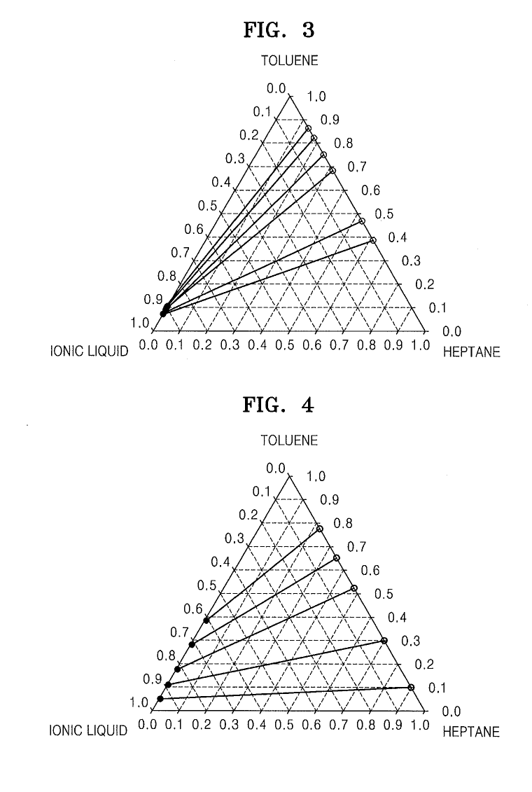

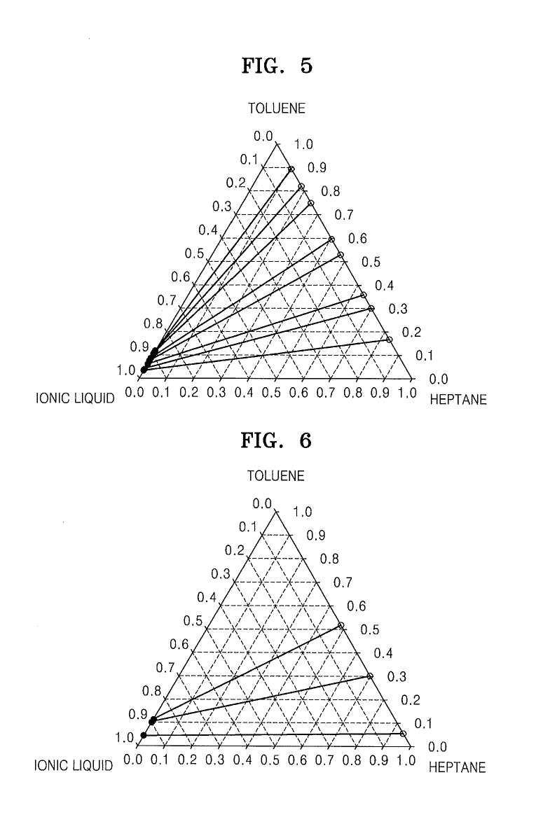

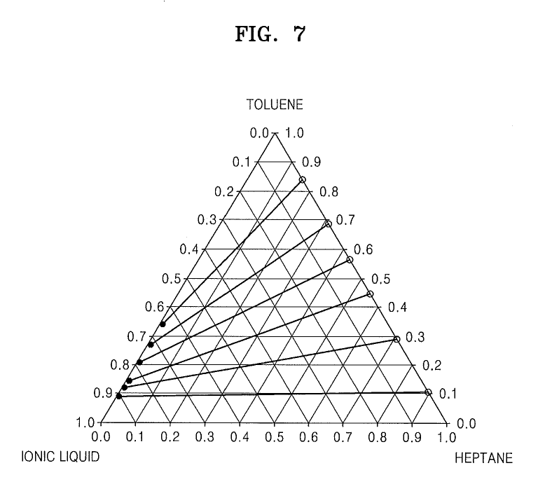

FIGS. 1 through 7 are triangular phase diagrams based on the composition of heptane/toluene/ionic liquid contained in the ionic liquid layer and the composition of heptane/toluene/ionic liquid contained in the heptane layer obtained in each example based on results of analysis by NMR and GC-MS. In FIGS. 1 through 7, black dots denote compositions of heptane/toluene/ionic liquid contained in the ionic liquid layer, white dots denote compositions of heptane/toluene/ionic liquid contained in the heptane layer, and lines connecting the black dots and white dots show that two dots connected by a line correspond to the same example.

Referring to FIGS. 1 through 7, layers are separated in the mixture containing heptane/toluene/ionic liquid regardless of a mixing ratio of toluene and heptane, heptane is not contained in the ionic liquid layer after the layers are separated, and the ionic liquid is not contained in the heptane layer. In other words, the ionic liquid does not dissolve in heptane, whereas toluene dissolves in the ionic liquid, and thus toluene is separated from heptane.

Also, referring to FIGS. 1 through 3, separating efficiency of toluene increases as separating temperature increases under the same conditions.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of separating an aromatic compound, the method comprising separating an aromatic compound from a mixture containing the aromatic compound and an aliphatic compound, wherein the separating is performed using a liquid-liquid extracting method using an ionic liquid as an extracting solvent.

2. The method of claim 1, wherein the ionic liquid comprises at least one type of compound selected from compounds represented by Formula 1 below:
(Cat₁⁺)(An₁⁻) (Formula 1)
wherein Cat₁⁺ comprises a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, wherein each cation comprises at least one substituted or unsubstituted C₂-C₂₀ alkenyl group; and An₁⁻ comprises an anion comprising at least one halogen atom.

3. The method of claim 2, wherein Cat₁⁺ comprises a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, wherein each cation comprises at least one group from the group consisting of a substituted or unsubstituted allyl group and vinyl group.

4. The method of claim 2, wherein Cat₁⁺ comprises a quaternary ammonium cation or quaternary phosphonium cation represented by Formula 2 below:
(R₁R₂R₃R₄Z⁺) (Formula 2)
wherein Z comprises nitrogen or phosphorous and R₁, R₂, R₃, and R₄ are each independently selected from the group consisting of a substituted or unsubstituted C₁-C₁₀ alkyl group, a substituted or unsubstituted C₃-C₁₀ cycloalkyl group, a substituted or unsubstituted C₂-C₂₀ alkenyl group, and a substituted or unsubstituted C₆-C₂₀ aryl group, wherein at least one of R₁, R₂, R₃, and R₄ comprises at least one group selected from the group consisting of a substituted or unsubstituted allyl group and a vinyl group.

5. The method of claim 1, wherein the ionic liquid comprises 1-butyl-3-propyl imidazolium bromide.

6. The method of claim 5, wherein the ionic liquid additionally comprises at least one compound selected from compounds represented by Formula 1′ below:
(Cat₂⁺)(An₂⁻) (1′)
wherein Cat₂⁺ comprises a cation selected from the group consisting of quaternary ammonium, quaternary phosphonium, sulfonium, imidazolium, pyridinium, pyrazolium, piperidinium, pyrrolidinium, triazolium, oxazolium, thiazolium, pyrazinium, pyrimidinium, and pyridazinium, and An₂⁻ comprises an anion comprising at least one halogen atom.

7. The method of claim 2, wherein An₁⁻ or An₂⁻ comprises an anion selected from the group consisting of F⁻, Cl⁻, Br⁻, I⁻, BF₄⁻, PF₆⁻, SbF₆⁻, AsF₆⁻, CF₃SO₃⁻, CF₃COO⁻, HCF₂CF₂SO₃⁻, CF₃HFCCF₂SO₃⁻, HCClFCF₂SO₃⁻, CF₃OCFHCF₂SO₃—, CF₃CF₂OCFHCF₂SO₃⁻, CF₃CFHOCF₂CF₂SO₃⁻, CF₂HCF₂OCF₂CF₂SO₃⁻, CF₂ICF₂OCF₂CF₂SO₃⁻, CF₃CF₂OCF₂CF₂SO₃⁻, (CF₂HCF₂SO₂)₂N⁻, (CF₃CFHCF₂SO₂)₂N⁻, CF₃CH₂CH₂COO⁻, (CF₃SO₂)₂N⁻, (CF₃SO₂)₂C⁻, (FSO₂)₂N⁻, (CF₃CF₂)₂(SO₂)₂N⁻, and PF₃(C₂F₅)₃⁻.

8. The method of claim 1, wherein the ionic liquid comprises at least one selected from the group consisting of 1-allyl-3-butyl imidazolium bromide, 1-allyl-3-butyl imidazolium chloride, 1-allyl-3-butyl imidazolium tetrafluoroborate, 1-allyl-3-butyl imidazolium hexafluorophosphate, 1-allyl-3-butyl imidazolium bis(trifluorosulfonyl)imide, 1,3-diallyl imidazolium bromide, 1,3-diallyl imidazolium chloride, 1,3-diallyl imidazolium tetrafluoroborate, 1,3-diallyl imidazolium hexafluoro phosphate, 1,3-diallyl imidazolium bis(trifluorosulfonyl)imide, 1-allyl-3-vinyl imidazolium bromide, 1-allyl-3-vinyl imidazolium chloride, 1-allyl-3-vinyl imidazolium tetrafluoroborate, 1-allyl-3-vinyl imidazolium hexafluoro phosphate, 1-allyl-3-vinyl imidazolium bis(trifluorosulfonyl)imide, 1-butyl-3-vinyl imidazolium bromide, 1-butyl-3-vinyl imidazolium chloride, 1-butyl-3-vinyl imidazolium tetrafluoroborate, 1-butyl-3-vinyl imidazolium hexafluoro phosphate, and 1-butyl-3-vinyl imidazolium bis(trifluorosulfonyl)imide.

9. The method of claim 1, wherein the mixture comprises at least 1 molar part of the aliphatic compound with respect to 100 molar parts of the aromatic compound.

10. The method of claim 6, wherein An₁⁻ or An₂⁻ comprises an anion selected from the group consisting of F⁻, Cl⁻, Br⁻, I⁻, BF₄⁻, PF₆⁻, SbF₆⁻, AsF₆⁻, CF₃SO₃⁻, CF₃COO⁻, HCF₂CF₂SO₃⁻, CF₃HFCCF₂SO₃⁻, HCClFCF₂SO₃⁻, CF₃OCFHCF₂SO₃—, CF₃CF₂OCFHCF₂SO₃⁻, CF₃CFHOCF₂CF₂SO₃⁻, CF₂HCF₂OCF₂CF₂SO₃⁻, CF₂ICF₂OCF₂CF₂SO₃⁻, CF₃CF₂OCF₂CF₂SO₃⁻, (CF₂HCF₂SO₂)₂N⁻, (CF₃CFHCF₂SO₂)₂N⁻, CF₃CH₂CH₂COO⁻, (CF₃SO₂)₂N⁻, (CF₃SO₂)₂C⁻, (FSO₂)₂N⁻, (CF₃CF₂)₂(SO₂)₂N⁻, and PF₃(C₂F₅)₃⁻.
